(19) United States
(12) Patent Application Publication
(10) Pub. No.: US 2012/0052030 A1
(43) Pub. Date: Mar. 1, 2012

(54) COSMETIC COMPOSITION COMBINING A SILICONE POLYAMIDE, A SILICONE RESIN AND AT LEAST 51% DYESTUFF

(75) Inventors: Padraig Mac Dermott, Meudon (FR); Gwenola Le Gars, Paris (FR)

(73) Assignee: L'OREAL, Paris (FR)

(21) Appl. No.: 13/257,323

(22) PCT Filed: Mar. 10, 2010
(86) PCT No.: PCT/EP2010/053032

§ 371 Date: Oct. 21, 2011

Related U.S. Application Data
(60) Provisional application No. 61/163,049, filed on Mar. 25, 2009.

(30) Foreign Application Priority Data
Mar. 18, 2009 (FR) 0951720

(51) Int. Cl. A61K 8/92; A61Q 1/02; A61Q 1/10; A61K 8/898

(52) U.S. Cl. 424/63

(57) ABSTRACT

The present invention relates to a cosmetic makeup or care composition comprising, in a physiologically acceptable medium, at least one fatty phase comprising: at least one silicone polyamide, at least one silicone resin, and at least 51% of dyestuff chosen from pigments, nacres and reflective particles, and mixtures thereof.

The present invention relates to a cosmetic eyeshadow composition comprising at least one liquid fatty phase comprising at least one silicone polyamide, at least one silicone resin and a high content of dyestuffs. The invention also relates to a process for making up the eyelids using the composition.

Eyeshadows are made from a suitable vehicle and various dyestuffs intended to give a certain colour to the makeup powder and, after their application, to the eyelids. They may be in aqueous or anhydrous form.

Many makeup compositions of this type are in the form of a compact powder generally comprising a fatty phase, conventionally known as a “binder”, and a pulverulent phase especially comprising pigments and/or fillers. They may also be in the form of a cream texture.

However, the eyeshadows with a powder texture that are currently commercially available have limited staying power over time, i.e. less than ten hours and generally between eight and ten hours. Similarly, eyeshadows with a cream texture have staying power of less than 16 hours. They thus need to be freshened in the course of the day.

Poor staying power over time may be reflected in particular by poor colour fastness over time. This poor staying power may be characterized by a modification in colour (change or fading of the colour) generally following an interaction with the sebum and/or sweat secreted by the skin. This obliges the user to freshen the makeup very regularly, which may be time consuming.

One object of the present invention is to propose a makeup that both has good staying power and is comfortable. In particular, the compositions according to the invention show, simultaneously, good staying power, i.e. longer than 20 hours, with a texture close to that of powders, and are also comfortable and do not transfer.

For the purposes of the invention, the term “staying power” means the fastness of the colour over time and also the absence of streaking (i.e. staying power of the product on the skin).

“Transfer-free” skin makeup compositions are compositions that have the advantage of forming a deposit that does not transfer, at least partly, onto supports with which they come into contact (glass, clothing, cigarette or fabrics).

The inventors have found, unexpectedly, that the use of a silicone resin in combination with a silicone polyamide and a high content of dyestuffs makes it possible to obtain compositions that have good staying power while at the same time being transfer-resistant, the compositions moreover being comfortable on application.

Consequently, according to a first aspect, the present invention relates to a cosmetic makeup or care composition comprising, in a physiologically acceptable medium, at least one fatty phase comprising:
at least one silicone polyamide,
at least one silicone resin, and
at least 51% of a dyestuff chosen from pigments, nacres and reflective particles, and mixtures thereof.

Preferably, the composition according to the invention comprising a fatty phase comprising at least one silicone polyamide is obtained by means of a specific process that affords a cosmetic composition of novel texture, namely a paste that is soft or in the form of chips, which has good elasticity. This texture allows a smooth, uniform film to be applied to keratin materials, and has good staying-power properties.

The deposit obtained with the composition according to the invention is uniform and shows improved staying power.

A subject of the present invention is also a process for making up or caring for keratin materials and especially the eyelids, in which a composition as defined previously is applied to the said keratin materials.

According to the invention, the composition is preferably solid.

The term “solid” characterizes the state of the composition at room temperature (25° C.) and at atmospheric pressure (760 mmHg).

Preferably, the composition according to the invention comprises less than 5% by weight and preferably less than 3% by weight of water relative to the total weight of the composition; preferably, the composition is free of water.

Preferably, the composition is an eyeshadow, an eyebrow composition, an eyeliner, a blusher or a face powder. Even more preferentially, the composition is a face powder or a blusher.

Silicone Polyamide

As indicated previously, the compositions according to the invention comprise at least one silicone polyamide.

The silicone polyamides of the composition are preferably solid at room temperature (25° C.) and atmospheric pressure (760 mmHg).

For the purposes of the invention, the term “polymer” means a compound containing at least 2 repeating units, preferably at least 3 repeating units and better still 10 repeating units.

The silicone polyamides of the composition of the invention may be polymers of the polyorganosiloxane type, for instance those described in documents U.S. Pat. No. 5,874,069, U.S. Pat. No. 5,919,441, U.S. Pat. No. 6,051,216 and U.S. Pat. No. 5,981,680. According to the invention, the silicone polymers may belong to the following two families:

(1) polyorganosiloxanes comprising at least two amide groups, these two groups being located in the polymer chain, and/or

(2) polyorganosiloxanes comprising at least two amide groups, these two groups being located on grafts or branches.

A) According to a first variant, the silicone polymers are polyorganosiloxanes as defined above in which the amide units are located in the polymer chain.

The silicone polyamides may be more particularly polymers comprising at least one unit corresponding to the general formula I:

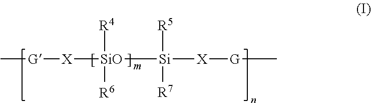

in which:

1) G′ represents C(O) when G represents —C(O)—NH—Y—NH—, and G′ represents —NH— when G represents —NH—C(O)—Y—C(O)—;

2) R⁴, R⁵, R⁶ and R⁷, which may be identical or different, represent a group chosen from:
linear, branched or cyclic, saturated or unsaturated, C₁-C₄₀ hydrocarbon-based groups, possibly containing in their chain one or more oxygen, sulfur and/or nitrogen atoms, and possibly being partially or totally substituted with fluorine atoms,
C₆-C₁₀ aryl groups, optionally substituted with one or more C₁-C₄ alkyl groups,
polyorganosiloxane chains possibly containing one or more oxygen, sulfur and/or nitrogen atoms;

3) the groups X, which may be identical or different, represent a linear or branched C₁-C₃₀ alkylenediyl group, possibly containing in its chain one or more oxygen and/or nitrogen atoms;

4) Y is a saturated or unsaturated, C₁-C₅₀ linear or branched divalent alkylene, arylene, cycloalkylene, alkylarylene or arylalkylene group, possibly comprising one or more oxygen, sulfur and/or nitrogen atoms, and/or bearing as substituent one of the following atoms or groups of atoms: fluorine, hydroxyl, C₃-C₈ cycloalkyl, C₁-C₄₀ alkyl, C₅-C₁₀ aryl, phenyl optionally substituted with 1 to 3 C₁-C₃ alkyl, C₁-C₃ hydroxyalkyl and C₁-C₆ aminoalkyl groups; or

5) Y represents a group corresponding to the formula:

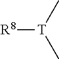

in which
T represents a linear or branched, saturated or unsaturated, C₃-C₂₄ trivalent or tetravalent hydrocarbon-based group optionally substituted with a polyorganosiloxane chain, and possibly containing one or more atoms chosen from O, N and S, or T represents a trivalent atom chosen from N, P and Al, and
R⁸ represents a linear or branched C₁-C₅₀ alkyl group or a polyorganosiloxane chain, possibly comprising one or more ester, amide, urethane, thiocarbamate, urea, thiourea and/or sulfonamide groups, which may possibly be linked to another chain of the polymer;

6) n is an integer ranging from 2 to 500 and preferably from 2 to 200, and m is an integer ranging from 1 to 1000, preferably from 1 to 700 and better still from 6 to 200.

According to the invention, 80% of the groups R⁴, R⁵, R⁶ and R⁷ of the polymer are preferably chosen from methyl, ethyl, phenyl and 3,3,3-trifluoropropyl groups. According to another embodiment, 80% of the groups R⁴, R⁵, R⁶ and R⁷ of the polymer are methyl groups.

According to the invention, Y can represent various divalent groups, furthermore optionally comprising one or two free valencies to establish bonds with other units of the polymer or copolymer. Preferably, Y represents a group chosen from:

a) linear C₁ to C₂₀ and preferably C₁ to C₁₀ alkylene groups,

b) C₃₀ to C₅₆ branched alkylene groups possibly comprising rings and unconjugated unsaturations,

c) C₅-C₆ cycloalkylene groups,

d) phenylene groups optionally substituted with one or more C₁ to C₄₀ alkyl groups,

e) C₁ to C₂₀ alkylene groups comprising from 1 to 5 amide groups,

f) C₁ to C₂₀ alkylene groups comprising one or more substituents chosen from hydroxyl, C₃ to C₈ cycloalkane, C₁ to C₃ hydroxyalkyl and C₁ to C₆ alkylamine groups,

g) polyorganosiloxane chains of formula:

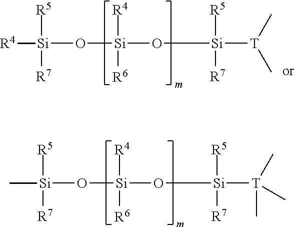

in which R⁴, R⁵, R⁶, R⁷, T and m are as defined above.

B) According to the second variant, the silicone polyamides may be polymers comprising at least one unit corresponding to formula (II):

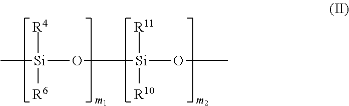

in which
R⁴ and R⁶, which may be identical or different, are as defined above for formula (I),
R¹⁰ represents a group as defined above for R⁴ and R⁶, or represents a group of formula —X-G″-R¹² in which X is as defined above for formula (I) and R¹² represents a hydrogen atom or a linear, branched or cyclic, saturated or unsaturated, C₁-C₅₀ hydrocarbon-based group optionally comprising in its chain one or more atoms chosen from O, S and N, optionally substituted with one or more fluorine atoms and/or one or more hydroxyl groups, or a phenyl group optionally substituted with one or more C₁-C₄ alkyl groups,

and G″ represents —C(O)NH— and —HN—C(O)—,
R¹¹ represents a group of formula —X-G″-R¹² in which X, G″ and R¹² are as defined above,
m₁ is an integer ranging from 1 to 998, and
m₂ is an integer ranging from 2 to 500.

According to the invention, the silicone polymer may be a homopolymer, that is to say a polymer comprising several identical units, in particular units of formula (I) or of formula (II).

According to the invention, it is also possible to use a polymer consisting of a copolymer comprising several different units of formula (I), that is to say a polymer in which at least one of the groups R⁴, R⁵, R⁶, R⁷, X, G, Y, m and n is different in one of the units. The copolymer may also be formed from several units of formula (II), in which at least one of the groups R⁴, R⁶, R¹⁰, R¹¹, m₁ and m₂ is different in at least one of the units.

It is also possible to use a polymer comprising at least one unit of formula (I) and at least one unit of formula (II), the units of formula (I) and the units of formula (II) possibly being identical to or different from each other.

According to one variant of the invention, it is also possible to use a silicone polyamide furthermore comprising at least one hydrocarbon-based unit comprising two groups capable of establishing hydrogen interactions, chosen from ester, sulfonamide, carbamate, thiocarbamate, urea, urethane, thiourea, oxamido, guanidino and biguanidino groups, and combinations thereof.

These copolymers may be block polymers or grafted polymers.

In formulae (I) and (II), the alkylene group representing X or Y can optionally contain in its alkylene part at least one of the following components:

1) one to five amide, urea, urethane or carbamate groups,

2) a C₅ or C₆ cycloalkyl group, and

3) a phenylene group optionally substituted with 1 to 3 identical or different C₁-C₃ alkyl groups.

In formulae (I) and (II), the alkylene groups may also be substituted with at least one component chosen from the group formed from:
a hydroxyl group,
a C₃-C₈ cycloalkyl group,
one to three C₁-C₄₀ alkyl groups,
a phenyl group optionally substituted with one to three C₁-C₃ alkyl groups,
a C₁-C₃ hydroxyalkyl group, and
a C₁-C₆ aminoalkyl group.

In these formulae (I) and (II), Y may also represent:

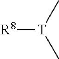

in which R⁸ represents a polyorganosiloxane chain and T represents a group of formula:

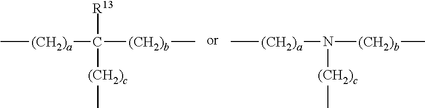

in which a, b and c are, independently, integers ranging from 1 to 10, and R¹⁶ is a hydrogen atom or a group such as those defined for R⁴, R⁵, R⁶ and R⁷.

In formulae (I) and (II), R⁴, R⁵, R⁶ and R⁷ preferably represent, independently, a linear or branched C₁ to C₄₀ alkyl group, preferably a CH₃, C₂H₅, n-C₃H₇ or isopropyl group, a polyorganosiloxane chain or a phenyl group optionally substituted with one to three methyl or ethyl groups.

As has been seen previously, the polymer may comprise identical or different units of formula (I) or (II).

Thus, the polymer may be a polyamide containing several units of formula (I) or (II) of different lengths, i.e. a polyamide corresponding to formula (III):

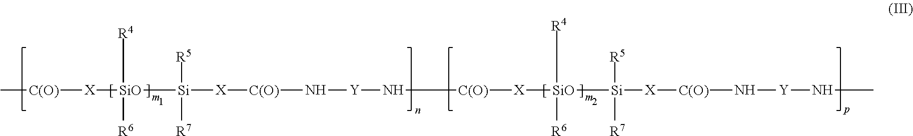

in which X, Y, n and R⁴ to R⁷ have the meanings given above, m₁ and m₂, which are different, are chosen in the range from 1 to 1000, and p is an integer ranging from 2 to 300.

In this formula, the units may be structured to form either a block copolymer, or a random copolymer or an alternating copolymer. In this copolymer, the units may be not only of different lengths, but also of different chemical structures, for example containing different groups Y. In this case, the polymer may correspond to formula (IV):

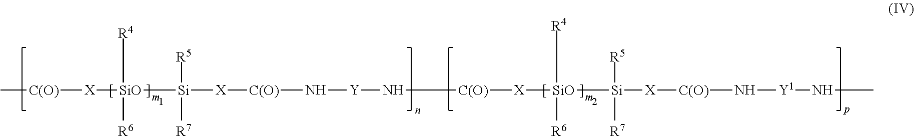

in which R⁴ to R⁷, X, Y, m₁, m₂, n and p have the meanings given above and Y¹ is different from Y but chosen from the groups defined for Y. As previously, the various units may be structured to form either a block copolymer, or a random copolymer or an alternating copolymer.

In this first embodiment of the invention, the silicone polymer may also consist of a grafted copolymer. Thus, the polyamide containing silicone units may be grafted and optionally crosslinked with silicone chains containing amide groups. Such polymers may be synthesized with trifunctional amines.

According to the invention, as has been seen previously, the siloxane units may be in the main chain or backbone of the polymer, but they may also be present in grafted or pendent chains. In the main chain, the siloxane units may be in the form of segments as described above. In the pendent or grafted chains, the siloxane units may appear individually or in segments.

According to one embodiment variant of the invention, a copolymer of silicone polyamide and of hydrocarbon-based polyamide, or a copolymer comprising units of formula (I) or (II) and hydrocarbon-based polyamide units, may be used. In this case, the polyamide-silicone units may be located at the ends of the hydrocarbon-based polyamide.

Advantageously, the composition comprises at least one polyamide/polydimethylsiloxane polymer, especially a polymer of general formula (I) with an index m of greater than 50, in particular greater than 75 and especially of about 100.

Advantageously, the silicone polyamide of formula (I) has a weight-average molecular mass ranging from 10 000 to 500 000 g/mol.

More preferably, X and Y independently represent a group chosen from linear C₁-C₂₀ and preferably C₁-C₁₀ alkylene groups.

As examples of polymers that may be used, mention may be made of one of the silicone polyamides obtained in accordance with Examples 1 to 3 of document U.S. Pat. No. 5,981,680.

Preferably, the nylon-611/dimethicone copolymer sold under the reference DC 2-8179 by Dow Corning is used as silicone polyamide.

The polymers and copolymers used in the composition of the invention advantageously have a temperature of transition from the solid state to the liquid state ranging from 45° C. to 190° C. Preferably, they have a temperature of transition from the solid state to the liquid state ranging from 70 to 130° C. and better still from 80° C. to 105° C.

The silicone polyamide may be present in the first composition in a total content ranging from 0.5% to 45% by weight relative to the total weight of the composition, preferably ranging from 1% to 30% by weight and better still ranging from 2% to 20% by weight relative to the total weight of the said composition.

Silicone Resin

The composition according to the invention comprises at least one silicone resin.

Examples of these silicone resins that may be mentioned include:
siloxysilicates, which may be trimethylsiloxy-silicates of formula [(CH₃)₃SiO]ₓ(SiO_{4/2})_{y} (units MQ) in which x and y are integers ranging from 50 to 80,
polysilsesquioxanes of formula (CH₃SiO_{3/2})ₓ (units T) in which x is greater than 100 and at least one of the methyl radicals of which may be substituted with a group R as defined above,
polymethylsilsesquioxanes, which are polysilsesquioxanes in which none of the methyl radicals is substituted with another group. Such polymethyl-silsesquioxanes are described in document U.S. Pat. No. 5,246,694, the content of which is incorporated by reference.

As examples of commercially available poly-methylsilsesquioxane resins, mention may be made of those sold:
by the company Wacker under the reference Resin MK, such as Belsil PMS MK: polymer comprising CH₃SiO_{3/2} repeating units (units T), which may also comprise up to 1% by weight of (CH₃)₂SiO_{2/2} units (units D) and having an average molecular weight of about 10 000,
by the company Shin-Etsu under the reference KR-220L, which are composed of units T of formula CH₃SiO_{3/2} and contain Si—OH (silanol) end groups, under the reference KR-242A, which comprise 98% of units T and 2% of dimethyl units D and contain Si—OH end groups, or under the reference KR-251, comprising 88% of units T and 12% of dimethyl units D and contain Si—OH end groups.

Siloxysilicate resins that may be mentioned include trimethyl siloxysilicate resins (TMS) optionally in the form of powders. Such resins are sold under the reference SR1000 by the company Momentive Performance Materials or under the reference TMS 803 by the company Wacker. Mention may also be made of trimethyl siloxysilicate resins sold in a solvent such as cyclomethicone, sold under the name KF-7312J by the company Shin-Etsu or DC 749 and DC 593 by the company Dow Corning.

Advantageously, the silicone resin, for instance the trimethyl siloxysilicate resin, is present in a content ranging from 0.5% to 30%, or better still from 1% to 25% or even better still from 5% to 25% relative to the total weight of the composition.

Preferably, the silicone resin, and especially the trimethyl siloxysilicate resin, is present in a ratio such that the silicone polyamide/silicone resin mass proportion is between 1/4 and 1 and preferably between 1/3 and 1.

Preferably, nylon-611/dimethicone is used as silicone polyamide and a trimethyl siloxysilicate resin is used as silicone resin.

Fatty Phase

The fatty phase of the composition according to the invention preferably comprises a liquid fatty phase comprising at least one oil.

In one preferred embodiment, the composition according to the invention comprises at least one volatile oil.

The composition may also comprise at least one non-volatile oil. According to one embodiment, the composition according to the invention is free of non-volatile oil.

The oil(s) may be present in a content ranging from 1% to 45% by weight and preferably from 5% to 40% by weight relative to the total weight of the composition.

For the purposes of the invention, the term “volatile oil” means an oil that is capable of evaporating on contact with the skin or the keratin fibre in less than one hour, at room temperature and atmospheric pressure. The volatile organic solvent(s) and volatile oils of the invention are volatile organic solvents and cosmetic oils that are liquid at room temperature, with a non-zero vapour pressure at room temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg), and more particularly ranging from 1.3 Pa to 1300 Pa (0.01 to 10 mmHg).

The term “non-volatile oil” means an oil that remains on the skin or the keratin fibre at room temperature and atmospheric pressure for at least several hours and that especially has a vapour pressure of less than 10⁻³ mmHg (0.13 Pa).

These oils may be hydrocarbon-based oils, silicone oils or fluoro oils, or mixtures thereof.

The term “hydrocarbon-based oil” means an oil mainly containing hydrogen and carbon atoms and optionally oxygen, nitrogen, sulfur or phosphorus atoms. The volatile hydrocarbon-based oils may be chosen from hydrocarbon-based oils containing from 8 to 16 carbon atoms, and especially branched C8-C16 alkanes, for instance C8-C16 isoalkanes of petroleum origin (also known as isoparaffins), for instance isododecane (also known as 2,2,4,4,6-pentamethyl-heptane), isodecane and isohexadecane, for example the oils sold under the trade names Isopar or Permethyl, branched C8-C16 esters and isohexyl neopentanoate, and mixtures thereof. Other volatile hydrocarbon-based oils, for instance petroleum distillates, especially those sold under the name Shell Solt by the company Shell, may also be used. The volatile hydrocarbon-based oils may also be chosen from linear C8-C16 alkanes. Examples of linear C8-C16 alkanes that may be mentioned include n-nonadecane (C9), n-decane (C10), n-undecane (C11), n-dodecane (C12), n-tridecane (C13), n-tetradecane (C14), n-pentadecane (C15) and n-hexadecane (C16), and mixtures thereof, and in particular the mixture of n-undecane (C11) and of n-tridecane (C13) sold under the reference Cetiol UT by the company Cognis.

According to one embodiment, a volatile linear alkane that is suitable for use in the invention may be chosen from n-nonadecane, n-undecane, n-dodecane and n-tridecane, and mixtures thereof.

In particular, a volatile linear alkane may be used in the form of an n-undecane/n-tridecane mixture.

Preferably, the n-undecane/n-tridecane weight ratio is in the range from 50/50 to 90/10, preferably from 60/40 to 80/20 and in particular ranging from 65/35 to 75/25.

In particular, a composition of the invention may comprise a mixture of n-undecane and n-tridecane in a 70/30 weight ratio.

Preferably, the volatile solvent is chosen from hydrocarbon-based volatile oils containing from 8 to 16 carbon atoms, and mixtures thereof.

Volatile oils that may also be used include volatile silicones, for instance volatile linear or cyclic silicone oils, especially those with a viscosity ≦8 centistokes (8×10⁻⁶ m²/s) and especially containing from 2 to 7 silicon atoms, these silicones optionally comprising alkyl or alkoxy groups containing from 1 to 10 carbon atoms. As volatile silicone oils that may be used in the invention, mention may be made especially of octamethylcyclotetrasiloxane, decamethyl-cyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof.

Mention may also be made of linear volatile alkyltrisiloxane oils of general formula (I):

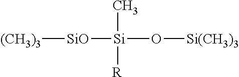

in which R represents an alkyl group containing from 2 to 4 carbon atoms, of which one or more hydrogen atoms may be substituted with a fluorine or chlorine atom.

Among the oils of general formula (I) that may be mentioned are:
3-butyl-1,1,1,3,5,5,5-heptamethyltrisiloxane,
3-propyl-1,1,1,3,5,5,5-heptamethyltrisiloxane, and
3-ethyl-1,1,1,3,5,5,5-heptamethyltrisiloxane,
   corresponding to the oils of formula (I) for which R is, respectively, a butyl group, a propyl group or an ethyl group.

Volatile fluoro solvents such as nonafluoro-methoxybutane or perfluoromethylcyclopentane may also be used.

Preferably, the composition comprises a content of volatile oil ranging from 1% to 45% by weight and preferably from 5% to 40% by weight relative to the total weight of the composition.

The composition may also comprise at least one non-volatile oil, chosen especially from non-volatile hydrocarbon-based oils and/or silicone oils and/or fluoro oils.

Non-volatile hydrocarbon-based oils that may especially be mentioned include:
hydrocarbon-based oils of plant origin, such as triglycerides consisting of fatty acid esters of glycerol, the fatty acids of which may have varied chain lengths from C4 to C24, these chains possibly being linear or branched, and saturated or unsaturated; these oils are especially wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin oil, sesame seed oil, marrow oil, rapeseed oil, blackcurrant oil, evening primrose oil, millet oil, barley oil, quinoa oil, rye oil, safflower oil, candlenut oil, passion flower oil and musk rose oil; or alternatively caprylic/capric acid triglycerides such as those sold by the company Stéarineries Dubois or those sold under the names Miglyol 810, 812 and 818 by the company Dynamit Nobel,
synthetic ethers containing from 10 to 40 carbon atoms;
linear or branched hydrocarbons of mineral or synthetic origin, such as petroleum jelly, polydecenes, hydrogenated polyisobutene such as Parleam, and squalane, and mixtures thereof;
synthetic esters such as oils of formula R1COOR2 in which R1 represents a linear or branched fatty acid residue containing from 1 to 40 carbon atoms and R2 represents an in particular branched hydrocarbon-based chain containing from 1 to 40 carbon atoms, on condition that R1+R2≧10, for instance purcellin oil (cetostearyl octanoate), isopropyl myristate, isopropyl palmitate, C12-C15 alkyl benzoate, hexyl laurate, diisopropyl adipate, isononyl isononanoate, 2-ethylhexyl palmitate, isostearyl isostearate, alkyl or polyalkyl octanoates, decanoates or ricinoleates such as propylene glycol dioctanoate; hydroxylated esters such as isostearyl lactate and diisostearyl malate; and pentaerythritol esters;
fatty alcohols that are liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol;
higher fatty acids such as oleic acid, linoleic acid or linolenic acid;
carbonates,
acetals,
citrates,
and mixtures thereof.

The non-volatile silicone oils that may be used in the compositions according to the invention may be non-volatile polydimethylsiloxanes (PDMSs), polydimethyl-siloxanes comprising alkyl or alkoxy groups, that are pendent and/or at the end of a silicone chain, the groups each containing from 2 to 24 carbon atoms, phenyl silicones, for instance phenyl trimethicones, phenyl dimethicones, phenyltrimethylsiloxydiphenyl-siloxanes, diphenyl dimethicones, diphenylmethyl-diphenyltrisiloxanes and 2-phenylethyl trimethylsiloxy-silicates.

The fluoro oils that may be used in the invention are, in particular, fluorosilicone oils, fluoro polyethers or fluorosilicones, as described in document EP-A-847 752.

Colouring Agent (or Dyestuff)

The colouring agent or dyestuff according to the invention is chosen from pigments, nacres and reflective particles, and mixtures thereof.

The colouring agent may be an organic pigment chosen from the materials hereinbelow, and mixtures thereof:
cochineal carmine,
organic pigments of azo dyes, anthraquinone dyes, indigoid dyes, xanthene dyes, pyrene dyes, quinoline dyes, triphenylmethane dyes and fluorane dyes.

Among the organic pigments, mention may be made especially of the D&C certified pigments known under the following names: D&C Blue No. 4, D&C Brown No. 1, D&C Green No. 5, D&C Green No. 6, D&C Orange No. 4, D&C Orange No. 5, D&C Orange No. 10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red No. 31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5, FD&C Yellow No. 6.

The chemical materials corresponding to each of the organic dyestuffs mentioned previously are mentioned in the publication “International Cosmetic Ingredient Dictionary and Handbook”, 1997 edition, pages 371 to 386 and 524 to 528, published by “The Cosmetic, Toiletries and Fragrance Association”, the content of which is incorporated into the present patent application by reference.

The colouring agent or dyestuff may also be chosen from nacres.

The term “nacre” should be understood as meaning coloured particles of any form, which may or may not be iridescent, especially produced by certain molluscs in their shell, or alternatively synthesized, and which have a colour effect via optical interference.

Examples of nacres that may be mentioned include nacreous pigments such as titanium mica coated with an iron oxide, mica coated with bismuth oxychloride, titanium mica coated with chromium oxide, and also nacreous pigments based on bismuth oxychloride. They may also be mica particles at the surface of which are superposed at least two successive layers of metal oxides and/or of organic dyestuffs.

The nacres may more particularly have a yellow, pink, red, bronze, orange, brown, gold and/or coppery colour or tint.

As illustrations of nacres that may be introduced as interference pigments into the first composition, mention may be made especially of the gold-coloured nacres sold especially by the company Engelhard under the name Brilliant gold 212G (Timica), Gold 222C (Cloisonne), Sparkle gold (Timica), Gold 4504 (Chromalite) and Monarch gold 233X (Cloisonne); the bronze nacres sold especially by the company Merck under the name Bronze fine (17384) (Colorona) and Bronze (17353) (Colorona) and by the company Engelhard under the name Super bronze (Cloisonne); the orange nacres sold especially by the company Engelhard under the name Orange 363C (Cloisonne) and Orange MCR 101 (Cosmica) and by the company Merck under the name Passion orange (Colorona) and Matte orange (17449) (Microna); the brown nacres sold especially by the company Engelhard under the name Nu-antique copper 340XB (Cloisonne) and Brown CL4509 (Chromalite); the nacres with a copper tint sold especially by the company Engelhard under the name Copper 340A (Timica); the nacres with a red tint sold especially by the company Merck under the name Sienna fine (17386) (Colorona); the nacres with a yellow tint sold especially by the company Engelhard under the name Yellow (4502) (Chromalite); the red nacres with a gold tint sold especially by the company Engelhard under the name Sunstone G012 (Gemtone); the pink nacres sold especially by the company Engelhard under the name Tan opale G005 (Gemtone); the black nacres with a gold tint sold especially by the company Engelhard under the name Nu antique bronze 240 AB (Timica), the blue nacres sold especially by the company Merck under the name Matte blue (17433) (Microna), the white nacres with a silvery tint sold especially by the company Merck under the name Xirona Silver, and the golden-green pink-orange nacres sold especially by the company Merck under the name Indian summer (Xirona), and mixtures thereof.

Still as examples of nacres, mention may also be made of particles comprising a borosilicate substrate coated with titanium oxide.

Particles having a glass substrate coated with titanium oxide are especially sold under the name Metashine MC1080RY by the company Toyal.

Finally, examples of nacres that may also be mentioned include polyethylene terephthalate flakes, especially those sold by the company Meadowbrook Inventions under the name Silver 1P0.004X0.004 (silver flakes).

Finally, the colouring agent according to the invention may be chosen from reflective particles.

The term “reflective particles” denotes particles whose size, structure, especially the thickness of the layer(s) of which they are made and their physical and chemical nature, and surface state allow them to reflect incident light. This reflection may, where appropriate, have an intensity sufficient to create at the surface of the composition or of the mixture, when it is applied to the support to be made up, points of overbrightness that are visible to the naked eye, i.e. more luminous points that contrast with their environment by appearing to sparkle.

The reflective particles may be selected so as not to significantly alter the coloration effect generated by the colouring agents with which they are combined, and more particularly so as to optimize this effect in terms of colour yield. They may more particularly have a yellow, pink, red, bronze, orange, brown, gold and/or coppery colour or tint.

These particles may have varied forms and may especially be in platelet or globular form, in particular spherical.

Irrespective of their form, the reflective particles may or may not have a multilayer structure, and, in the case of a multilayer structure, for example at least one layer of uniform thickness, especially a reflective material.

When the reflective particles do not have a multilayer structure, they may be composed, for example, of metal oxides, especially titanium or iron oxides obtained synthetically.

When the reflective particles have a multilayer structure, they may comprise, for example, a natural or synthetic substrate, especially a synthetic substrate at least partially coated with at least one layer of a reflective material, especially of at least one metal or metallic material. The substrate may be a monomaterial, multimaterial, organic and/or mineral substrate.

More particularly, it may be chosen from glasses, ceramics, graphite, metal oxides, aluminas, silicas, silicates, especially aluminosilicates and borosilicates, and synthetic mica, and mixtures thereof, this list not being limiting.

The reflective material may comprise a layer of metal or of a metallic material.

Reflective particles are described especially in documents JP-A-09188830, JP-A-10158450, JP-A-10158541, JP-A-07258460 and JP-A-05017710.

Again as an example of reflective particles comprising a mineral substrate coated with a layer of metal, mention may also be made of particles comprising a silver-coated borosilicate substrate.

Particles with a silver-coated glass substrate, in the form of platelets, are sold under the name Microglass Metashine REFSX 2025 PS by the company Toyal. Particles with a glass substrate coated with nickel/chromium/molybdenum alloy are sold under the name Crystal Star GF 550 and GF 2525 by this same company.

Particles comprising a metallic substrate such as silver, aluminium, iron, chromium, nickel, molybdenum, gold, copper, zinc, tin, manganese, steel, bronze or titanium, may also be used, the said substrate being coated with at least one layer of at least one metal oxide such as titanium oxide, aluminium oxide, iron oxide, cerium oxide, chromium oxide or silicon oxides, and mixtures thereof.

Examples that may be mentioned include aluminium powder, bronze powder or copper powder coated with SiO₂ sold under the name Visionaire by the company Eckart.

Preferably, the colouring agent is chosen from: organic pigments such as
the pigments certified D&C by the Food & Drug Administration as listed in the section “Color Additives—Batch Certified by the U.S. Food and Drug Administration” of the CTFA; mention may be made especially of Blue 1 and 4, Brown 1, Ext. Violet 2, Ext. Yellow 7, Green 3, 5, 6 and 8, Orange 4, 5, 10 and 11, Red 4, 6, 7, 17, 21, 22, 27, 28, 30, 36 and 40, Violet 2, Yellow 5, 6, 7, 8, 10 and 11,
   mineral pigments such as:
iron oxide, titanium oxide, zirconium oxide, cerium oxide, zinc oxide, iron oxide or chromium oxide,
ferric blue, manganese violet, ultramarine blue, pink or violet, chromium hydrate, chromium hydroxide or bismuth oxychloride,
   nacres such as:
mica coated with titanium oxide, mica coated with titanium oxide and iron oxide, and mica coated with an amino acid such as lauroyl lysine,
polyethylene terephthalate flakes,
sericite,
and mixtures thereof,
   reflective particles such as:
particles comprising a borosilicate substrate coated with a metallic layer.

The dyestuff(s) chosen from pigments, nacres and reflective particles according to the invention are present in a content of greater than or equal to 51% by weight, preferably ranging from 52% to 90% by weight, preferably in a content of greater than or equal to 55% by weight, ranging, for example, from 55% to 80% by weight, relative to the total weight of the composition.

Fillers

The eyeshadow according to the invention may comprise at least one filler that may be organic or mineral, and of spherical or lamellar form.

The fillers may be chosen from fillers known as incompactable fillers such as:
silica microspheres, especially of open porosity or, preferably, hollow silica microspheres, such as the products “Silica Beads SB 700/HA” or “Silica Beads SB 700” from the company Maprecos; these microspheres may be impregnated with a cosmetic active agent;
microporous polymer microspheres, which have a structure similar to that of a sponge; they generally have a specific surface area of at least 0.5 m²/g and in particular of at least 1 m²/g, the said specific surface area having no upper limit other than that resulting from the practical possibility of making microspheres of very high porosity: the specific surface area may be, for example, up to 1000 m²/g or even more. Illustrations of these microspheres that may be mentioned include acrylic polymer microspheres, such as those made of crosslinked acrylate copolymer “Polytrap 6603 Adsorber” from the company RP Scherer, and those made of polymethyl methacrylate “Micropearl M 100” from the company SEPPIC;
polyurethane powder, such as the powdered copolymer of hexamethylene diisocyanate and of trimethylol hexyl lactone sold under the names Plastic Powder D-400 and T-7 by the company Toshiki;
polymer microcapsules bearing only one closed cavity forming a reservoir, which may contain a liquid, especially a cosmetic active agent; they are prepared via known processes, such as those described in U.S. Pat. No. 3,615,972 and EP-A 0 56 219. They may be made, for example, of polymers or copolymers of ethylenically unsaturated acid, amine or ester monomers, of urea-formaldehyde polymers or of vinylidene chloride polymers or copolymers; by way of example, mention may be made of microcapsules made of methyl acrylate or methacrylate polymers or copolymers, or alternatively of copolymers of vinylidene chloride and of acrylonitrile; among these polymers, mention will be made especially of those containing 20-60% by weight of units derived from vinylidene chloride, 20-60% by weight of units derived from acrylonitrile and 0-40% by weight of other units such as units derived from an acrylic and/or styrene monomer; crosslinked acrylic polymers or copolymers may also be used;
elastomeric crosslinked organopolysiloxane spherical powders, described especially in document JP-A-02 243 612, such as those sold under the name “Trefil Powder E-506C” by the company Dow Corning;
the carnauba wax microbeads sold under the name Microcare 350® by the company Micro Powders and the paraffin wax microbeads sold under the name Microease 114S® by the company Micro Powders;
metal soaps in powder form. Among these soaps, mention may be made especially of metal soaps of fatty acids containing from 12 to 22 carbon atoms and in particular those containing from 12 to 18 carbon atoms. The metal of the metal soap may especially be zinc or magnesium. The fatty acid may be chosen especially from lauric acid, myristic acid, stearic acid and palmitic acid. The metal soaps that may be used include zinc laurate, magnesium stearate, magnesium myristate and zinc stearate, and mixtures thereof;
talcs or hydrated magnesium silicates, especially in the form of particles generally less than 40 μm in size;
micas or aluminosilicates of varied composition that are especially in the form of flakes from 2 to 200 μm and preferably 5-70 μm in size and from 0.1 to 5 μm and preferably 0.2-3 μm in thickness, these micas possibly being of natural origin (for example muscovite, margarite, roscoelite, lipidolite or biotite) or of synthetic origin;
clays such as sericites, which belong to the same chemical and crystalline class as muscovite;
kaolin or hydrated aluminium silicate, which is especially in the form of particles of isotropic forms generally less than 30 μm in size;
boron nitrides;
powders of tetrafluoroethylene polymers, such as Ceridust 9205 F from the company Clariant;
precipitated calcium carbonate, especially in the form of particles greater than 10 μm in size;
magnesium carbonate and magnesium hydrogen carbonate;
hydroxyapatite;
powders of non-expanded synthetic polymers, such as polyethylene, polyesters (for example polyethylene isophthalate or terephthalate) and polyamides (for example Nylon), in the form of particles less than 50 μm in size;
powders of spheronized, crosslinked or non-crosslinked synthetic polymers, for instance polyamide powders such as poly-β-alanine powder or Nylon powder, for example Orgasol powder from the company Atochem, polyacrylic acid or polymethacrylic acid powder, powders of polystyrene crosslinked with divinylbenzene, and silicone resin powders, and
powders of organic materials of natural origin, for instance starches, especially corn starch, wheat starch or rice starch;
   and mixtures thereof.

The fillers may be present in a content ranging from 0.1% to 30% by weight and preferably ranging from 1% to 10% by weight relative to the total weight of the composition.

The composition may comprise other ingredients (adjuvants) usually used in cosmetics, such as preserving agents, cosmetic active agents, moisturizers, UV screening agents, thickeners, surfactants and fragrances.

Needless to say, a person skilled in the art will take care to select the optional adjuvants(s) added to the composition according to the invention such that the advantageous properties intrinsically associated with the composition in accordance with the invention are not, or are not substantially, adversely affected by the envisaged addition.

According to another aspect, the invention also relates to a cosmetic assembly comprising:

i) a container delimiting a compartment, the said compartment being closed by a closing member; and

ii) a composition in accordance with the invention placed inside the said compartment.

The container may be of any suitable form. It may especially be a bottle, a tube, a jar, a case, a can, a sachet or a box.

The closing member may be in the form of a removable stopper, a lid, a cover, a tear-off strip or a capsule, especially of the type comprising a body fixed to the container and a cap articulated on the body. It may also be in the form of a member that selectively closes the container, especially a pump or a valve, for instance a clapper.

Preferably, the composition is inside a leaktight container.

Preparation Process

The composition according to the invention is preferably prepared according to the following procedure:
a) a formulation base comprising the silicone polyamide and the MQ resin is prepared:
   This base may also comprise one or more oils, especially volatile oils, and also a gelling agent; and
b) this formulation base is mixed with a fatty phase, especially a volatile or non-volatile silicone oil, using a blender or an extruder.

Thus, the present patent application also relates to a process for preparing a composition according to the present invention, comprising at least one step of controlled shear, preferably continuously, of all or part of the fatty phase structured with the silicone polyamide, advantageously at a temperature greater than or equal to the solid-liquid transition temperature of the silicone polymer (phase b)).

Preferably, the process comprises at least one step of controlled shear of all of the ingredients of the composition, advantageously at a temperature greater than or equal to the solid-liquid transition temperature of the silicone polymer.

Any equipment or equipment combination for controlling a mechanical action that will blend the product gradually as it is prepared, such as processes functioning continuously of scraped-surface exchanger type, or preferably of blender/twin-screw extruder type (referred to for simplicity as “extruder” in the rest of the present document) is suitable for use in the process according to the invention.

Extruders of blender/twin-screw extruder type are preferably used, these extruders being composed of the following elements:
at least two jackets independently temperature-regulated to a temperature ranging from 10° C. to 300° C.,
two co-rotating axles composed of screw elements, each element having a shape that provides the desired mixing function in the corresponding temperature zone,
devices for metering and introducing the various phases,
a variable-speed motor, for modulating the intensity of the shear as a function of the spin speed of the screws.

When the process according to the invention is performed in a blender/extruder, the various ingredients may be incorporated at different temperatures in the course of the blending during cooling, at a temperature that is compatible with their stability.

As equipment for performing the invention, mention will be made especially, without limiting the invention to these machines, of the models BC-21 and BC-45 from the company Clextral, or the model Prism Eurolab from the company ThermoRheo.

The examples that follow are given as non-limiting illustrations of the present invention. The percentages are weight percentages.

### EXAMPLES 1 AND 2

### Eyeshadows

### Preparation of the Formulation Base:

| | | |
|---|---|---|
| A | Isododecane | 35.43 |
| A | Trimethyl siloxysilicate resin (SR1000 from | 24.44 |
| | Momentive Performance Materials) | |
| B | Nylon-611/Dimethicone copolymer (Dow Corning 2- | 17.29 |
| | 8179 Gellant from Dow Corning) | |
| B | Isopropyl alcohol | 1.24 |
| C | Isododecane | 6.5 |
| C | Trimethyl siloxysilicate resin (SR1000 from | 6.5 |
| | Momentive Performance Materials) | |
| C1 | Smectite: Modified magnesium silicate in | 7.36 |
| | isododecane (Bentone Gel ISD V from Elementis) | |
| D | 5 μm amorphous silica microspheres (Sunsphere H | 1.24 |
| | 51 from AGC SI-TECH) | |

| | |
|---|---|
| Total | 100 |

The MQ resin is mixed with part of the isododecane, and the mixture is allowed to swell (phases A and C separately).

The isopropyl alcohol is mixed with the silicone polyamide (phase B), and phase B obtained is then mixed with the Bentone C1 with Moritz blending, in order to obtain a composition.

Finally, phases A and C, and the filler D, are mixed with the composition obtained, with Moritz stirring or vigorous Rayneri stirring.

The formulation base is thus obtained.

Preparation of Eyeshadows:

| | Example 1 | Example 2 |
|---|---|---|
| Ingredient | (%) | (%) |
| 5 μm amorphous silica | 0.45 | 0.25 |
| microspheres (Sunsphere H 51 | | |
| from AGC SI-TECH) | | |
| Smectite: Modified magnesium | 2.65 | 1.47 |
| silicate in isododecane | | |
| (Bentone Gel ISD V from | | |
| Elementis) | | |
| Iron oxides | | 6.4 |
| Mica-titanium oxide-brown, | 18.75 | 18.75 |
| black iron oxides (Cloisonne | | |
| Nu Antique Bronze from | | |
| Engelhard) | | |
| Mica-titanium oxide (Flamenco | 31.25 | 31.25 |
| Orange 320 C from Engelhard) | | |
| Lauroyl lysine-coated mica | 5 | 1.5 |
| (Mearlmica Treated SVA from | | |
| Engelhard) | | |
| Hexamethylene diisocyanate/- | 5 | 2.1 |
| trimethylhexyl lactone copolymer | | |
| powder containing silica | | |
| 10-15 μm (Plastic Powder D 400 | | |
| from Toshiki Pigment) | | |
| Phenyl trimethylsiloxytrisiloxane | 4 | |
| (viscosity: 20 cSt) (Dow | | |
| Corning 556 Cosmetic Grade | | |
| Fluid from Dow Corning) | | |
| Cyclohexyldimethylsiloxane | | 20 |
| (viscosity: 8 cSt) (Dow | | |
| Corning 246 Fluid from Dow | | |
| Corning) | | |
| Trimethyl siloxysilicate resin | 11.14 | 6.19 |
| (SR1000 from Momentive | | |
| Performance Materials) | | |
| Nylon-611/Dimethicone | 6.22 | 3.45 |
| copolymer (Dow Corning 2-8179 | | |
| Gellant from Dow Corning) | | |
| Isopropyl alcohol | 0.45 | 0.25 |
| Isododecane | 15.1 | 8.39 |

The compositions of Examples 1 and 2 are obtained according to the following protocol:
the formulation base is mixed with the silicone oil (i.e. Dow Corning 556 or 246);
the fillers, nacres and pigments are then added, and the mixture is placed in an extruder.

The compositions of Examples 1 and 2 were each evaluated by a panel of 9 women for three days.

After daily application of each composition for 3 days, the staying power was judged as being particularly good: the makeup effect remains as at the time of application. Moreover, the compositions are comfortable.

1: A cosmetic composition comprising, in a physiologically acceptable medium, at least one fatty phase comprising:
at least one silicone polyamide;
at least one silicone resin; and
at least 51% of dyestuff selected from the group consisting of pigments, nacres, reflective particles, and mixtures thereof,
wherein the composition is suitable for cosmetic makeup or care.

2: The composition of claim 1, comprising less than 5% by weight of water relative to the total weight of the composition.

3: The composition of claim 1, wherein a total silicone polyamide content is from 0.5% to 45% of the total weight of the composition.

4: The composition of claim 1, wherein the silicone polyamide comprises at least one unit of formula (I): 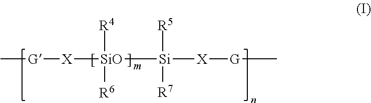
wherein:
1) G′ represents C(O) when G represents —C(O)—NH—Y—NH—, and G′ represents —NH— when G represents —NH—C(O)—Y—C(0)-;
2) R⁴, R⁵, R⁶ and R⁷, which may be identical or different, represent a group selected from the group consisting of:
linear, branched or cyclic, saturated or unsaturated, C₁-C₄₀ hydrocarbon-based groups, optionally comprising in their chain one or more oxygen, sulfur and/or nitrogen atoms, and optionally substituted, partially or totally, with fluorine atoms,
C₆-C₁₀ aryl groups, optionally substituted with one or more C₁-C₄ alkyl groups,
polyorganosiloxane chains optionally comprising one or more oxygen, sulfur and/or nitrogen atoms;
3) the groups X, which may be identical or different, represent a linear or branched C₁-C₃₀ alkylenediyl group, optionally comprising in its chain one or more oxygen and/or nitrogen atoms;
4) Y is a saturated or unsaturated, C₁-C₅₀ linear or branched divalent alkylene, arylene, cycloalkylene, alkylarylene or arylalkylene group, optionally comprising one or more oxygen, sulfur and/or nitrogen atoms, and/or bearing at least one substituent selected from the group consisting of fluorine, hydroxyl, C₃-C₈ cycloalkyl, C₁-C₄₀ alkyl, C₅-C₁₀ aryl, phenyl optionally substituted with 1 to 3 C₁-C₃ alkyl, C₁-C₃ hydroxyalkyl and C₁-C₆ aminoalkyl groups; or
5) Y represents a group of formula (II): 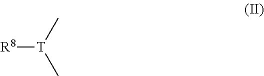
wherein
T represents a linear or branched, saturated or unsaturated, C₃-C₂₄ trivalent or tetravalent hydrocarbon-based group optionally substituted with a polyorganosiloxane chain, and optionally comprising at least one atom selected from the group consisting of O, N and S, or T represents a trivalent atom selected from the group consisting of N, P and Al, and
R⁸ represents a linear or branched C₁-C₅₀ alkyl group or a polyorganosiloxane chain, optionally comprising one or more ester, amide, urethane, thiocarbamate, urea, thiourea and/or sulfonamide groups, which may optionally be linked to another chain of the polyamide; and
6) n is an integer ranging from 2 to 500, and m is an integer ranging from 1 to 1000.

5: The composition of claim 4, wherein X and/or Y represent an alkylene group comprising in its alkylene part at least one of the following elements:
1) one to five amide, urea, urethane or carbamate groups;
2) a C₅ or C₆ cycloalkyl group; and
3) a phenylene group optionally substituted with 1 to 3 identical or different C₁-C₃ alkyl groups,
and/or substituted with at least one selected from the group consisting of:
a hydroxyl group;
a C₃-C₈ cycloalkyl group;
one to three C₁-C₄₀ alkyl groups;
a phenyl group optionally substituted with one to three C₁-C₃ alkyl groups;
a C₁-C₃ hydroxyalkyl group; and
a C₁-C₆ aminoalkyl group.

6: The composition of claim 4, wherein R⁴, R⁵, R⁶ and R⁷ independently represent a linear or branched C₁ to C₄₀ alkyl group, a polyorganosiloxane chain or a phenyl group optionally substituted with 1 to 3 methyl or ethyl groups.

7: The composition of claim 4, comprising at least one polymer of formula (I) with an index m of greater than 50.

8: The composition of claim 1, wherein the silicone resin is a siloxysilicate resin.

9: The composition of claim 1, wherein the silicone resin represents 0.5% to 30% relative to the total weight of the composition.

10: The composition of claim 1, wherein the silicone resin is present in a ratio such that a silicone polyamide/silicone resin mass proportion is between 1/4 and 1.

11: The composition of claim 1, wherein the silicone polyamide is a nylon-611/dimethicone copolymer and the silicone resin is a trimethyl siloxysilicate resin.

12: The composition of claim 1, further comprising at least one volatile oil.

13: The composition of claim 1, wherein the at least one volatile oil represents from 1% to 45% of the total weight of the composition.

14: The composition of claim 12, wherein the at least one volatile oil is selected from the group consisting of hydrocarbon-based volatile oils comprising from 8 to 16 carbon atoms, and mixtures thereof.

15: A process for making up keratin materials, comprising applying the composition of claim 1.

16: The composition of claim 1, wherein the composition is free of water.

17: The composition of claim 4, wherein R⁴, R⁵, R⁶ and R⁷ independently represent a CH₃, C₂H₅, n-C₃H₇ or isopropyl, a polyorganosiloxane chain or a phenyl group optionally substituted with 1 to 3 methyl or ethyl groups.

18: The composition of claim 1, wherein the silicone resin is a trimethyl siloxysilicate resin.

19: The composition of claim 1, comprising a total silicone polyamide content from 1% to 30% of the total weight of the composition.

20: A process for making up eyelids, comprising applying the composition of claim 1 to the eyelids.
